(19) United States
(12) Patent Application Publication
(10) Pub. No.: US 2014/0163412 A1
(43) Pub. Date: Jun. 12, 2014

(54) MYOGRAPHY METHOD AND SYSTEM

(71) Applicant: Vibrado Technologies, Inc., Sunnyvale, CA (US)

(72) Inventors: Quinn A. Jacobson, Sunnyvale, CA (US); Cynthia Kuo, Mountain View, CA (US)

(73) Assignee: Vibrado Technologies, Inc., Sunnyvale, CA (US)

(21) Appl. No.: 14/103,264

(22) Filed: Dec. 11, 2013

Related U.S. Application Data
(60) Provisional application No. 61/735,916, filed on Dec. 11, 2012.

(51) Int. Cl. A61B 5/0488; A61B 5/11; A61B 5/00

(52) U.S. Cl. CPC A61B 5/0488; A61B 5/7203; A61B 5/11
USPC 600/546

(57) ABSTRACT

A myography system and method can compensate for background noise in order to analyze data indicative of muscle contraction. Compensating for background noise may include any of: removing a model of the actual background noise from frequency data obtained from a myography sensor, identifying which myography sensor from among a plurality of myography sensors is located at a muscle likely undergoing contraction, and narrowing the analysis to searching for the type of muscular contraction (e.g., concentric, isometric, or eccentric) that is likely to be occurring. A model of the actual background noise can be obtained through use of myography sensors on different parts of the moving body. The muscles which are likely to be under contraction and the types of muscle contraction that are likely to be occurring at those muscles can be identified through use of motion capture devices, such as imaging devices and inertial measurement units.

### CROSS-REFERENCE TO RELATED APPLICATION

This application claims the benefit of U.S. Provisional Application No. 61/735,916, filed Dec. 11, 2012, which is incorporated herein by reference in its entirety and for all purposes.

### FIELD OF THE INVENTION

The invention relates, in general, to signal processing and more particularly to analysis of data related to muscle activity.

### BACKGROUND OF THE INVENTION

The study of muscular contraction is called myography. A muscle contraction occurs when the central nervous system sends signals to a motor neuron in the spinal cord, and the motor neuron is activated. The motor neuron releases acetylcholine, a neurotransmitter that triggers a response from the muscle fiber. Muscular contractions can be detected by measuring electrical, vibrational, or acoustic signals indicative of muscle activity. See, for example, U.S. Patent Application Publication Nos. 2010/0262042 (entitled “Acoustic Myography Systems and Methods”), 2010/0268080 (entitled “Apparatus and Technique to Inspect Muscle Function”), 2012/0157886 (entitled “Mechanomyography Signal Input Device, Human-Machine Operating System and Identification Method Thereof”), 2012/0188158 (entitled “Wearable Electromyography-based Human-Computer Interface), 2013/0072811 (entitled “Neural Monitoring System”), and 2013/0289434 (entitled “Device for Measuring and Analyzing Electromyography Signals”).

Electromyography measures electrical impulses directly in the muscle fibers or indirectly on the surface of the skin. Acoustic myography detects low frequency sounds, which are mostly inaudible, created during muscle activity. Mechanomyography detects small vibrations of a muscle when it contracts. The vibrations are virtually invisible to the naked eye.

Myography is useful for applications such as physical therapy and sports training Myography allows a therapist or trainer to observe the muscle activity and fatigue of a person's muscles. Information obtained from the observations can be used to access progress in strengthening the muscles or in the efficiency of using muscles. However, the utility of conventional myography is generally limited. Conventional myography does not measure concentric and eccentric muscular contractions (use of muscles with movement). Measurements in conventional myography are confined to isometric muscular contractions (use of muscles without movement) because data from sensors are generally unreliable during body movement. In electromyography and mechanomyography, signals from the muscle(s) of interest are easily overwhelmed by signals generated during body movement. When a person moves his or her body, the energy of muscles other than the muscle(s) of interest and the energy of the moving body as a whole are transmitted throughout the body, which results in a huge amount of background energy. The signals which one wants to detect with myography are relatively weak and may not be recognized in the presence of the background energy or noise.

For example, one may wish to analyze the activity of certain muscles while a person is running From the perspective of mechanomyography, each time the runner's feet hits the ground a large amount of vibrational energy is sent through the runner's entire body. There is also vibrational energy that is produced when the runner subsequently pushes off her foot after the foot hits the ground and when the runner swings or pumps her arms as she counterbalances the forces in her legs. All this background vibrational energy travels through the body and covers the entire frequency range, including the frequencies that mechanomyograhpy observes for muscle analysis. Thus, the frequencies of interest for analysis can be masked or rendered unrecognizable by the background vibrational energy.

Accordingly, there is a need for a myography method and system that is useful during natural body movements. Also, there is a need for a myography method and system that measures both concentric and eccentric muscular contractions while the body is moving, in addition to isometric muscular contractions.

### SUMMARY OF THE INVENTION

Briefly and in general terms, the present invention is directed to a myography method, a myography system, and a non-transitory computer readable medium for performing myography.

In aspects of the present invention, a method comprises obtaining frequency data from each of a plurality of myography sensors on a body in motion, wherein the myography sensors include a target myography sensor at a target muscle on the body and one or more myography sensors located at other parts of the body, the motion of the body produces background noise, and the frequency data from the target myography sensor includes the background noise and data on muscle contraction of the target muscle. The method further comprises analyzing the data on muscle contraction of the target muscle, including compensating for the background noise.

In aspects of the present invention, a system comprises a plurality of myography sensors, and a processor configured to obtain frequency data from each of the plurality of myography sensors while the myography sensors are on a body in motion. The processor is further configured to compensate for background noise produced by body motion to analyze data on muscle contraction from a target muscle.

In aspects of the present invention, a non-transitory computer readable medium has a stored computer program embodying instructions, which when executed by a computer, causes the computer to perform myography. The computer readable medium comprises instructions for obtaining frequency data from each of a plurality of myography sensors on a body in motion, wherein the myography sensors include a target myography sensor at a target muscle on the body and one or more myography sensors located at other parts of the body. The computer readable medium further comprises instructions for analyzing data on muscle contraction of the target muscle, including compensating for background noise which is produced by the motion of the body and is present in the frequency data together with the data on muscle contraction.

The features and advantages of the invention will be more readily understood from the following detailed description which should be read in conjunction with the accompanying drawings.

### BRIEF DESCRIPTION OF THE DRAWINGS

The drawings are not necessarily to scale.

FIG. 1 is an internal view of an arm showing a myography sensor for detecting signals indicative of contraction of a biceps muscle.

FIG. 2 is a perspective diagram showing an inertial measurement unit.

FIG. 3 is a block diagram showing an inertial measurement unit.

FIG. 4 is partial internal view of a portion of a body showing a myography sensor and processor for detecting signals indicative of contraction of a muscle.

FIG. 5 is a graph showing time domain measurements of acceleration amplitude indicative of muscle vibrations, the measurements provided by the myography sensor of FIG. 4.

FIG. 6 is a graph showing a frequency domain representation of the measurements of FIG. 5.

FIG. 7 is a block diagram showing a myography system having myography sensors attached to a body and in communication with a processor.

FIG. 8 is a time domain view of signals indicative of muscle contractions provided by the myography sensors of FIG. 7.

FIG. 9 is a frequency domain view of signals indicative of muscle contractions provided by the myography sensors of FIG. 7.

FIG. 10 is model of background noise derived from signals indicative of muscle contractions provided by the myography sensors of FIG. 7.

FIG. 11 is a graph showing frequency data provided by one of the myography sensors of FIG. 7 after the model of background noise has been removed.

FIG. 12 is a flow diagram of an exemplary myography method showing steps for removing at least some of the actual background noise using a background noise model.

FIG. 13 is a block diagram showing a myography system having myography sensors attached to a body and in communication with a processor configured to construct a background noise model.

FIG. 14 is a flow diagram of an exemplary myography method showing steps for removing at least some of the actual background noise and for targeting analysis to muscles likely to be in contraction based on a model of biomechanical movement of the body obtained by at least one motion capture device.

FIG. 15 is a block diagram showing a myography system including myography sensors and motion capture devices in communication with a processor.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### DETAILED DESCRIPTION OF EXEMPLARY EMBODIMENTS

While some of the exemplary embodiments herein are described in relation to vibrations and mechanomyography, the method and means for analysis of the present invention can apply to acoustic myography by processing acoustic signals indicative of muscle activity and can apply to electromyography by processing electrical signals obtained from muscle fibers or on the surface of the skin. Also, although some exemplary embodiments are described in the context of a person, the method and means for analysis of the present invention can apply equally to human and animal bodies. As used herein the term “body” refers to any type of body that can be subjected to myography unless specified otherwise.

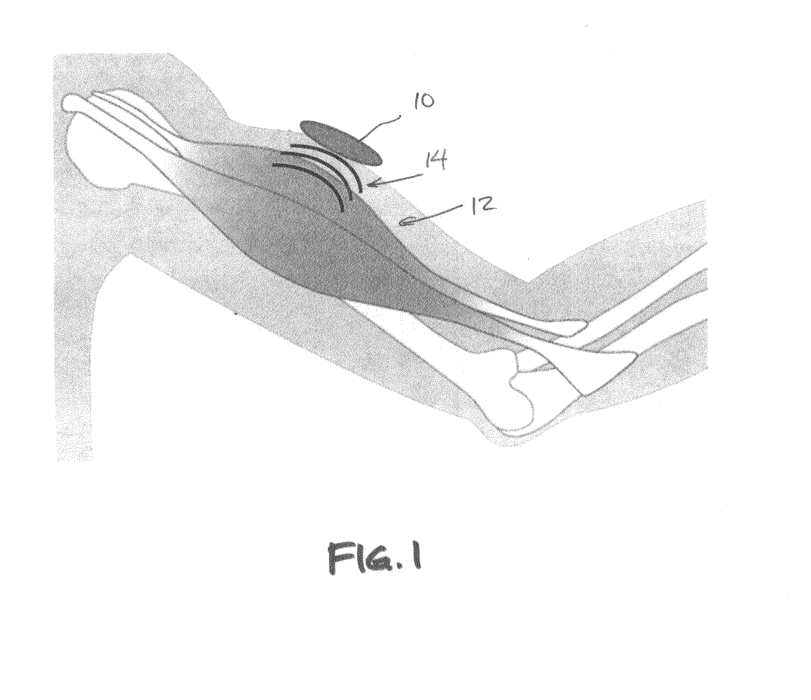

Referring now in more detail to the exemplary drawings for purposes of illustrating exemplary embodiments of the invention, wherein like reference numerals designate corresponding or like elements among the several views, there is shown in FIG. 1 myography sensor 10 placed near biceps muscle 12 of a person's arm for the purpose of analyzing activity of that muscle. The signal from myography sensor 10 includes signals indicative of muscle contraction, such as signals corresponding to electrical impulses from the muscle, signals corresponding to vibrations from the muscle, or signals corresponding to acoustics from the muscle. The signal from myography sensor 10 is sampled (i.e., obtained) over a period of time and then the samples are processed to obtain signal levels at different frequencies. Obtaining signal levels at different frequencies involves transforming the samples which were taken sequentially over a period of time (time domain measurements) to the frequency domain. The transformation to the frequency domain can be accomplished by well-known mathematical processes, such as Fourier transforms. Muscle activity can then be inferred by applying one or more heuristic processes to the frequency domain representation of the measurements taken by myography sensor 10.

In some embodiments, myography sensor 10 is a mechanomyography sensor configured to detect mechanical vibrations 14 in the range of a few vibrations per second through a few hundred vibrations per second. Examples of sensors which can detect such vibrations include without limitation piezoelectric sensors and microelectromechanical systems (MEMS) accelerometers. Data from myography sensor 10 is processed to obtain the amount of vibration occurring at different frequencies, to which one or more heuristic process can then be applied to infer muscle activity.

Data from additional myography sensors at other locations on the body can optionally be used to obtain a model of background noise, which can then be removed from the measurements in the frequency domain representation of signals from myography sensor 10. Also, motion capture sensors (also referred to as “motion capture devices” herein) for sensing the biomechanical motion of the body can be used in addition to the various myography sensors in order to identify the muscle or muscles that are likely to have been activated and/or to determine how the muscle or muscles are working (e.g., working by concentric, isometric, or eccentric contraction).

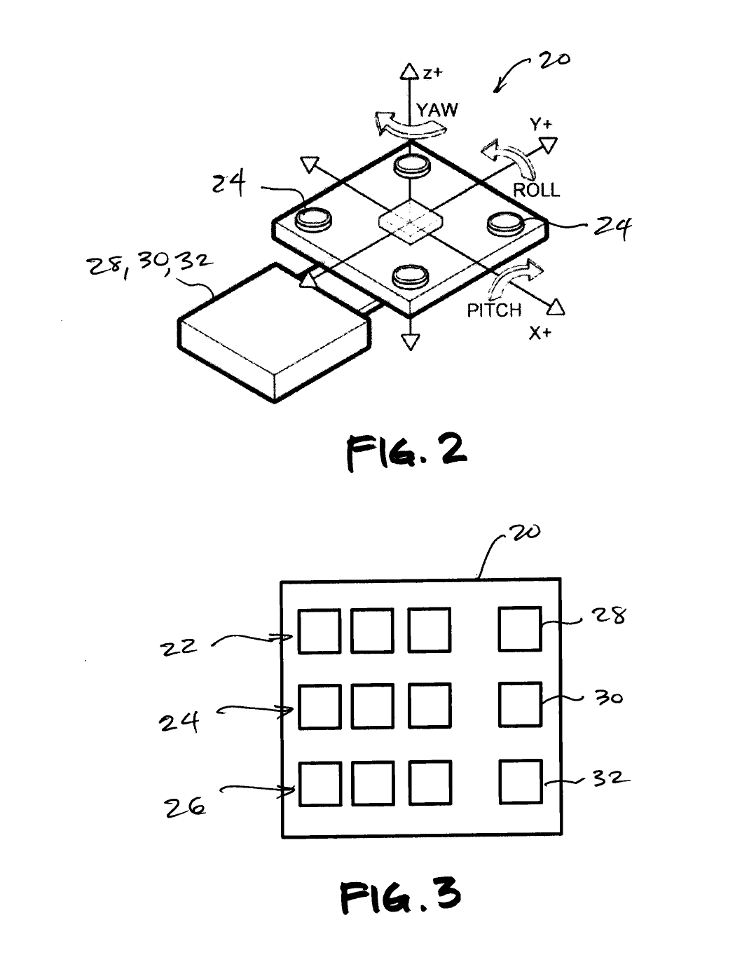

A non-limiting example of a motion capture sensor is an inertial measurement unit. FIGS. 2 and 3 show exemplary inertial measurement unit 20 which is configured to provide information on its orientation, velocity, and acceleration. Inertial measurement unit 20 uses a plurality of sensors that measure orientation and motion. The sensors include gyroscopes 22, accelerometers 24, and magnetometers 26. Gyroscopes 22 are configured to measure the rate and direction of rotation. Accelerometers 24 are configured to measure linear acceleration. Magnetometers 26 are configured to detect direction relative to magnetic north pole. Inertial measurement unit 20 may also include other electronic components, such as processor 28 and memory device 30 to receive, process, and store measurement data from gyroscopes 22, accelerometers 24, and magnetometers 26. Processor 28 may be a digital device that includes one or more semiconductor chips. Memory device 30 is configured for any one or a combination of volatile and non-volatile data storage. Inertial measurement unit 20 may also include communication device 32 for transmitting the data to an external device for evaluation and storage.

Inertial measurement unit 20 can be manufactured to be very small using advances in microelectromechanical systems (MEMS). Such advances need not be described herein as they are known by persons skilled in the art. For example, many smart phones may now include miniature sensors, such as gyroscopes, accelerometers, and magnetometers, which are capable of providing the functions of an inertial measurement unit. See, for example, U.S. Patent Application Publication Nos. 2011/0221664 (entitled “View Navigation on Mobile Device”), 2010/0167686 (entitled “Method of Power Management for a Handheld Mobile Computing Communication Device”), and 2009/0133499 (entitled “Accelerometer Module for Use with a Touch Sensitive Device”). With miniaturization, it is possible to place multiple inertial measurement units on different parts of a body in order to capture the motion of the body.

The use of motion capture sensors in combination with multiple myography sensors allows myography to be used while the body is moving. Data from the various sensors help to narrow the search for the relatively weak signals associated with myography. This can be accomplished by performing one or a combination of the following: (1) looking at sensors which are measuring the same type of measurement as the myography in order to characterize the background energy and remove the background energy, and (2) looking at biomechanics data to identify the type of body motion and to narrow the search for signals associated with myography.

Using the characteristics of body motion increases the utility and accuracy of the muscle activity measurements made by myography sensors. Using the characteristics of body motion also enables observation and correction of body motions during real world activities. For example, the ability to track the intensity of muscle contractions over time could allow a coach to monitor an athlete's calf muscle during a long-distance run to detect muscle fatigue.

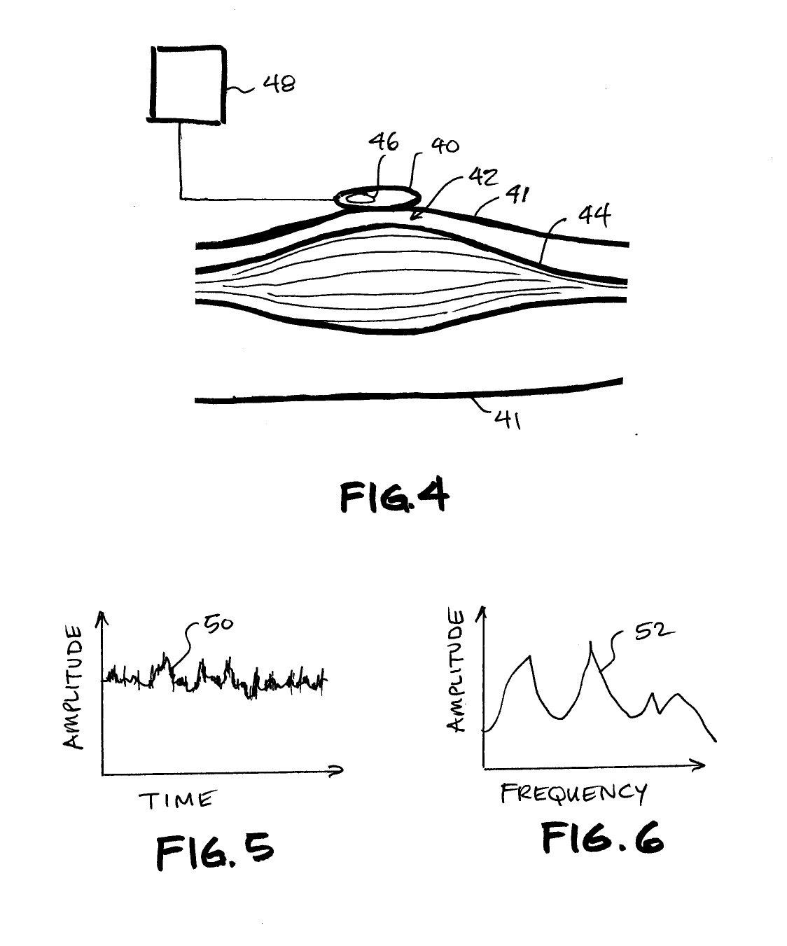

In an exemplary embodiment, myography sensor 40 is placed on skin 41 above muscle belly 42 of target muscle 44, as shown in FIG. 4. The term “target muscle” refers to the muscle that one is interested analyzing. The term “muscle belly” refers to the thickest part of the muscle. Target muscle 44 can be in a person's arm, leg, or other part of the anatomy.

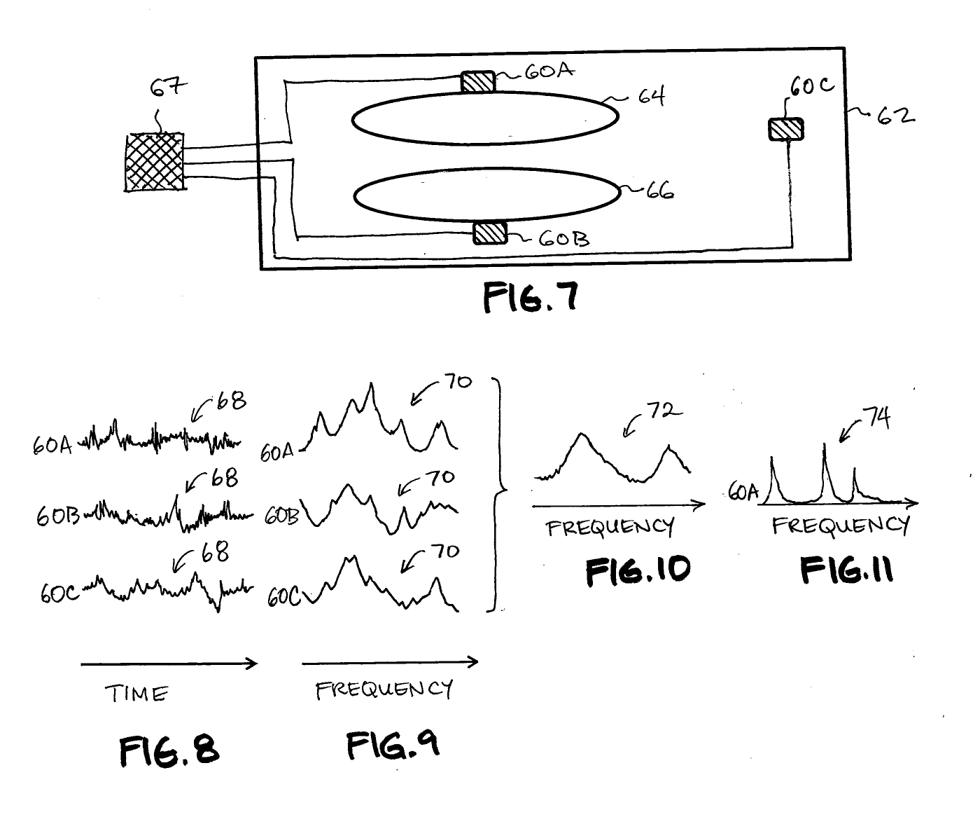

Myography sensor 40 is a mechanomyography sensor that includes accelerometer 46 configured to detect the vibration signal of target muscle 44. Data from accelerometer 46 is sampled (i.e., obtained) by processor 48 at a frequency greater than or equal to the Nyquist rate for the muscle vibrations. Processor 48 then filters the data from accelerometer 46. Filtering may include removing invalid or corrupt data packets. After filtering, what remains are acceleration measurements 50 of the muscle vibration taken sequentially over a period of time, as shown in FIG. 5. Acceleration measurements 50 are in the time domain. Next, acceleration measurements 50 are processed with a Fourier transform to obtain frequency spectrum 52 that reveals the frequencies of the muscle vibrations and the amount of energy at each frequency as shown in FIG. 6. Frequency spectrum 52 is the frequency domain representation of acceleration measurements 50 and is referred to as “frequency data” herein. If the body was in motion when data from accelerometer 46 was obtained by processor 48, frequency spectrum 52 will be dominated by the energy of the body motion. Additional processing can optionally be performed, as described below, to address the problem presented by the body motion energy (also referred to as “background energy” and “background noise” herein) in frequency spectrum 52.

An exemplary way to address the problem presented by background energy in the frequency domain is to construct a model of the background energy and remove it from frequency spectrum 52. This can be performed by placing multiple myography sensors 60 at different locations on body 62 to detect signals from muscles, as shown in FIG. 7. Myography sensor 60A is placed over target muscle 64, which is the muscle one wishes to analyze. Myography sensor 60A is referred to as the target myography sensor. Myography sensor 60B is placed on the opposite (antagonist) muscle 66 on the same limb or body region. Myography sensor 60C is placed on a different limb or body region. Additional myography sensors can optionally be used, so that there are at least two myography sensors placed over target muscle 64, and/or at least two myography sensors placed over antagonist muscle 66, and/or at least two myography sensors placed on different limbs or body regions. The use of additional myography sensors can increase accuracy by providing additional data points from which to construct the model of background energy.

Processor 67 is communicatively coupled to each of myography sensors 60A-60C. Communication between processor 67 and the myography sensors can be accomplish wirelessly and/or using wires. For example, communication may be in the form of any one or a combination of electric signals through wires and electromagnetic signals through space.

Processor 67 is configured to perform any one or a combination of steps described herein for removing background energy, for inferring what muscles are likely to be activated, and for determining how the muscle is working if it is active (e.g., working by concentric, isometric, or eccentric contraction). Processor 67 can be part of an electronic computer capable of executing, in accordance with a computer program stored on a non-transitory computer readable medium, any one or a combination of the steps described herein for removing background energy, for inferring what muscles are likely to be activated, and for determining how the muscle is working if it is active. The non-transitory computer readable medium may comprise any one or a combination of instructions for removing background energy, for inferring what muscles are likely to be activated, and for determining how the muscle is working if it is active.

Processor 67 can include one or more electronic semiconductor chips. Processor 67 may also include one or more memory devices for any one or a combination of volatile and non-volatile data storage. Processor 67 can be located at a distance away from body 62, or processor 67 may be attached to body 62. Processor 67 can be a personal computer, such as a laptop computer or a desktop computer. Processor 67 can be an embedded digital device. As an embedded digital device, processor 67 can be embedded within one or more of myography sensors and/or motion capture sensors. Processor 67 can be a mobile digital device, such as a smart phone or tablet.

As shown in FIG. 8, data 68 from myography sensors 60A-60C are obtained in parallel over the same time period. Data 68 are in the time domain. Data 68 represent vibration in the case of mechanomyography, electrical signals from muscles in the case of electromyography, or acoustic signals in the case of acoustic myography. Data 68 from each sensor is converted into the frequency domain using a Fourier transform, which results in frequency spectrum 70 shown in FIG. 9. Each frequency spectrum 70 is the frequency domain representation of corresponding data 68 from one of myography sensors 60A-60C.

Next, frequency spectra 70 (referred to herein as “frequency data”) from the multiple myography sensors are compared to look for commonality to produce model 72 of the background noise shown in FIG. 10. The frequencies that exhibit high energy in all, a majority, or a threshold number of the myography sensors can be presumed to be part of the background noise and are then included in background noise model 72. Background noise model 72 is intended to be approximation of the actual background noise and may not match the actual background noise perfectly. Background noise model 72 is then used to try to remove the actual background noise from frequency data 70 provided by target myography sensor 60A. Removing at least some of the actual background noise will make signals 74 local to target myography sensor 60A, which were produced by target muscle 64, stand out and be easier to recognize in the frequency domain, as shown in FIG. 11.

It is to be understood that the curves shown for time-domain data 68 in FIG. 8 and frequency-domain data 70 in FIG. 9 are simplified representations and are not intended to be mathematically accurate. Also, the curve shown for background noise model 72 in FIG. 10 is a simplified representation and is not intended to a mathematically precise representation of a particular method of constructing background noise model 72 from frequency-domain data 70. Further, the curve for frequency spectra 74 in FIG. 11 is a simplified representation and is not intended to show a mathematically accurate result of removing background noise model 72 from frequency spectra 70 of target myography sensor 60A. It is also to be understood that the curves shown in FIGS. 8-11 may represent a series of discrete amplitudes that form individual data points that are connected by the illustrated curve.

In general, the frequencies that exhibit high energy in all, a majority, or a threshold number of myography sensors can be presumed to be part of the background noise and are then included to background noise model 72. The comparison to look for commonality in the frequency data can be performed in different ways to produce background noise model 72. Examples of methods to compare frequency data include without limitation any one or combination of the following techniques.

A technique to compare frequency data may include computing the average for a set of myography sensors. In the example of FIGS. 7-9, frequency spectra 70 from myography sensors 60A, 60B and 60C can be averaged to produce background noise model 72. The average represents an approximation of the actual background noise. Averaging may include computing, at each frequency in frequency spectra 70, the mean value of the amplitudes measured by myography sensors 60A, 60B and 60C. The mean values at the various frequencies collectively form a mean curve which may be used as background noise model 72.

A technique to compare frequency data may include normalizing the frequency spectra 70 from myography sensors 60A, 60B and 60C, and then averaging the normalized frequency spectra to produce background noise model 72. Normalizing includes scaling each frequency spectrum to obtain modified (i.e., normalized) frequency spectra in which the energy is the same for all myography sensors 60A, 60B and 60C. In the case of mechanomyography, each frequency spectrum 70 can be scaled up or down as needed so that the total amount of vibrational energy is fixed, i.e., so that the total amount of vibrational energy in the normalized frequency spectra is the same for each one of myography sensors 60A, 60B and 60C. Thereafter, the average of the normalized frequency spectra is computed to obtain background noise model 72.

Normalization handles the possibility that the amplitude scale of the signal (either a vibrational signal during mechanomyography, electrical signal during electromyography, or acoustic signal during acoustic myography) measured by a myography sensor can vary from that of other myography sensors due to internal variations from sensor to sensor, due to external variables such as variations in how the myography sensors are attached to the body, and/or other reasons. For example, one of the myography sensors may be attached in a manner that dampens the overall energy that the myography sensor observes. Normalizing could include scaling up the frequency spectrum of the dampened myography sensor and/or scaling down the frequency spectra of the other myography sensors. The normalized frequency spectra can be used with the other techniques below.

Another technique to compare frequency data may include applying a weighted average to frequency spectra 70 so that the impact or effect of each one of myography sensors 60A, 60B and 60C is weighted differently during construction of background noise model 72.

The weight applied to each frequency spectrum 70 can be based on distance from the target myography sensor. However, there can be a tricky tradeoff in advantages and disadvantages that should be considered. On one hand, myography sensors (e.g., sensor 60B) close to the target myography sensor (e.g., sensor 60A) on the target muscle will see more similar background noise so frequency spectrum 70 from myography sensor 60B can be given more weight when computing the average among myography sensors 60A, 60B, and 60C. That is, background noise observed by myography sensor 60B will be more similar to that observed by target myography sensor 60A as compared to that observed by myography sensor 60C located on a different limb or body region. On the other hand, sensor 60B may also see at least some of the same signal of interest as sensor 60A (e.g., at least some of the signal indicating activation of target muscle 64, such as vibrational energy in the case of mechanomyography). Thus, if all the high energy frequencies common to both sensors 60A and 60B are interpreted as background noise, the signal of interest could be mistakenly discarded as background noise.

The weight can also be based on deviation or mean square deviation of frequency spectra 70. The deviation or mean square deviation can be used to deciding whether to include or omit frequency spectrum 70 of a particular myography sensor. In addition or alternatively, the deviation or mean square deviation can be used to select a weight to be applied to frequency spectrum 70 of a particular myography sensor.

Still another technique to compare frequency data may include computing a median curve for a set of myography sensors. In the example of FIGS. 7-9, the median curve can be computed from frequency spectra 70 of myography sensors 60A, 60B and 60C. The median curve represents an approximation of the actual background noise. Creating the median curve may include computing, at each frequency in frequency spectra 70, the median value of the amplitudes measured by myography sensors 60A, 60B and 60C. The median values at the various frequencies collectively form a median curve which may be used as background noise model 72.

Another technique to compare frequency data may include computing a trimmed mean curve for a set of myography sensors. The trimmed mean curve is computed from the frequency spectra of all myography sensors on the body, with some data points being discarded. For example, creating the trimmed mean curve may include computing, at each frequency in frequency spectra 70, the trimmed mean value. The trimmed mean value is the mean of the amplitudes measured by all myography sensors except the myography sensors with the highest and lowest amplitudes, and/or except for the myography sensors which exhibit amplitudes below a minimum threshold level, and/or except for the myography sensors which exhibit amplitudes above a maximum threshold level. The trimmed mean values at the various frequencies collectively form a trimmed mean curve which may be used as background noise model 72.

In an exemplary embodiment, data from at least one target myography sensor (e.g., sensor 60A) on the target muscle, at least one near myography sensor (e.g., sensor 60B) located near the target muscle, and at least one far myography sensor (e.g., sensor 60C) are collected simultaneously. The data includes measurements taken sequentially in time. The data are filtered, such as by removing bad or corrupt data packets, and run through a Fourier transform to obtain a frequency spectrum for each myography sensor. The frequency spectrum (also referred to as “frequency data” herein) is a frequency domain representation of the measurements taken sequentially in time. The level of background noise is determined by finding common frequencies with high levels of energy in the power spectral density. The term “common frequencies” refer to frequencies in common among the various myography sensors. For example, a frequency that has a high level of energy in the power spectral density of the frequency spectra of all, a majority, or a threshold percentage of myography sensors may be interpreted as background noise and then be used to construct background noise model 72. On the other hand, another frequency that has a high level of energy in the power spectral density of the frequency spectra of a few myography sensors but a low level of energy in the power spectral density of the frequency spectra of many other myography sensors may not be interpreted as background noise and then omitted from background noise model 72.

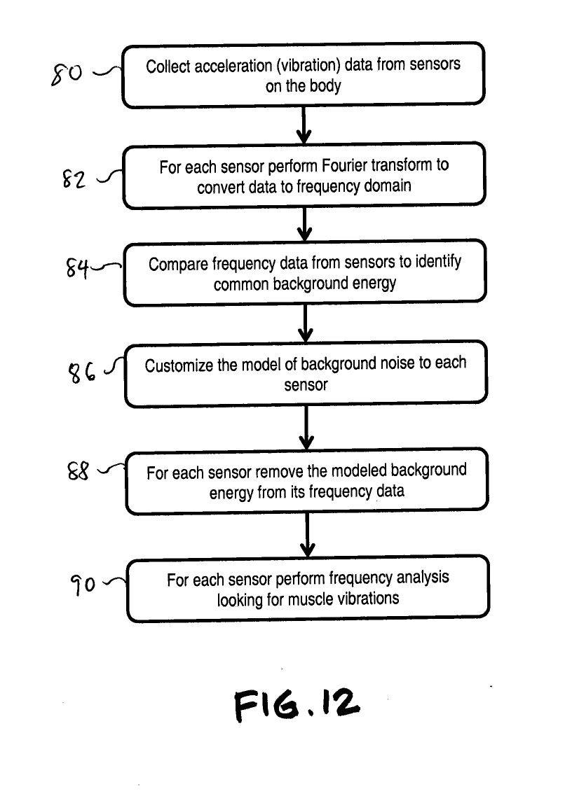

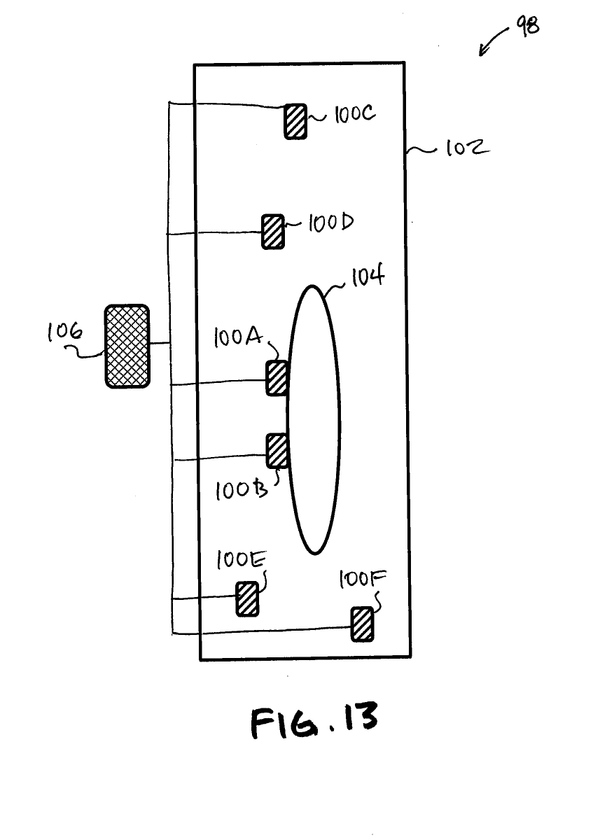

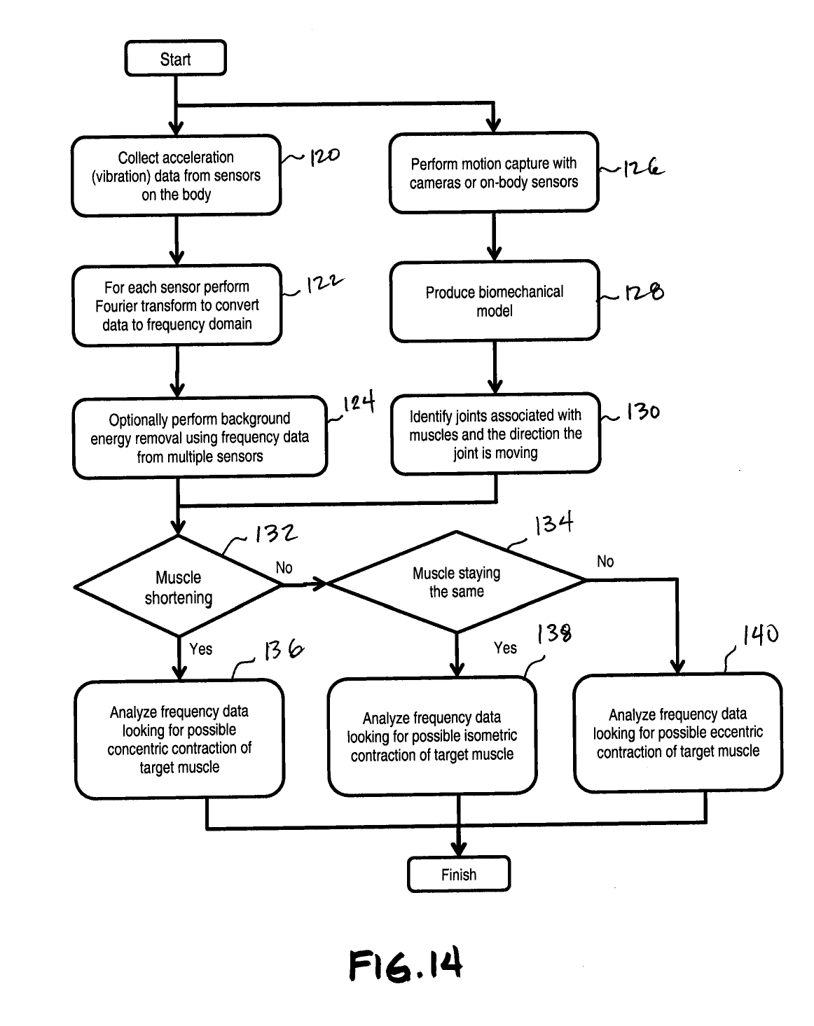

FIG. 12 illustrates an exemplary method for removing background noise or background energy when multiple myography sensors are placed on a body. Some of the sensors are placed near target muscles. Target muscles are those muscles in which it is desired to identify muscle activity. Although the method will be described with reference to devices previously described herein, it will be appreciated that other devices or groups of devices may be used to perform the method.

In block 80, acceleration data is collected (e.g., by processor 67) from the myography sensors (e.g., sensors 60A-60C). The acceleration data represents vibrations originating from the target muscle and other parts of a moving body. The acceleration data is collected for periods of time to acquire sufficient data that will allow frequency analysis to be performed. The period of time could, for example, be half second with samples taken every few milliseconds, or less than one second with samples taken every few milliseconds. Other time periods and sampling rates can be used.

In block 82, a Fast Fourier Transform (FFT) is performed (e.g., by processor 67) on the acceleration data from each myography sensor to produce frequency data representing what frequencies of vibration were observed and the amount of energy at each frequency.

In block 84, the frequency data from different myography sensors are compared to identify the background vibrational energy common to all, a majority, or a threshold percentage of myography sensors. This can be performed by computing an unweighted average, or by a more complicate processes, to identify and summarize commonality, such as described above in connection with background noise model 72 in FIG. 10. Exemplary processes to identify and summarize commonality include any one or a combination of computing a unweighted average of frequency data from the myography sensors, computing a weighted average of frequency data from the myography sensors, normalizing frequency data from the myography sensors, using deviation or mean square deviation to determine weights applied when computing a weighted average, using deviation or mean square deviation to determine whether frequency data from a particular myography sensor is discarded, computing a median of frequency data from the myography sensors, and computing a trimmed mean of frequency data from the myography sensors.

In block 86, the model of background noise is computed (e.g., by processor 67) from identification of common background vibrational energy in block 84. The model of background noise may be computed differently for each target myography sensor.

As shown for example in FIG. 13, system 98 includes multiple mechanomyography sensors 100 attached to body 102 to detect muscle vibrations while the body is moving. Two target mechanomyography sensors 100A and 100B are placed over target muscle 104 which is to be the subject of analysis. Additional mechanomyography sensors 100C-100F are located at various distances away from target muscle 104 to help construct a model of the background noise. As previously discussed, there are advantages and disadvantages in using mechanomyography sensors located close to the target muscle and in using mechanomyography sensors located far from the target muscle. Closer mechanomyography sensors are more likely to see similar background energy but are also more likely to also pick up vibrations of interest from the target muscle that should not be identified as background energy. Accordingly in an exemplary scenario, the background noise model for target mechanomyography sensor 100A may be constructed from a weighted average of frequency data from all mechanomyography sensors with the greatest weight applied to the frequency data from mechanomyography sensor 100D. On the other hand, the background noise model for target mechanomyography sensor 100B may be constructed from a weighted average of frequency data from all mechanomyography sensors with the greatest weight applied to the frequency data from mechanomyography sensor 100E.

It will be appreciated that there can be more than one target muscle, each with its own target myography sensor or sensors. For example, one may wish to study activation of the calf, quadriceps, and hamstring muscles on the same leg over the same time period, or one may wish to study activation of the calf muscle in each of the left and right legs over the same time period. The target myography sensor for one of the target muscles could have a background noise model that differs from that of the target myography sensor for the other target muscles.

Referring again to FIG. 12, in block 88 the background noise model is removed (e.g., by processor 67) from the frequency data of each myography sensor. Removal can be performed by subtracting the background noise model from the frequency data. The result of the removal is referred to herein as the “resultant frequency data.” This could be performed by subtracting, at each frequency, the amplitude of the background noise model from the amplitude of the frequency data of each myography sensor.

In block 90, for each of the target myography sensors, analysis of the resultant data is performed (e.g., by processor 67) to look for indications of muscle activity, such as vibrations indicative of muscle contraction in the case of mechanomyography, electrical signals indicative of muscle contraction in the case of electromyography, or acoustic signals indicative of muscle contraction in the case of acoustic myography.

The process steps of FIG. 12 can also be performed, for example, by processor 106 communicatively coupled to mechanomyography sensors 100A-100F in FIG. 13. Processor 106 can be configured like processor 67 in FIG. 7. Processor 106 can be part of an electronic computer capable of executing, in accordance with a computer program stored on a non-transitory computer readable medium, any one or a combination of the steps described herein for performing any one or a combination of the steps of FIG. 12. The non-transitory computer readable medium may comprise instructions for performing any one or a combination of the steps of FIG. 12.

As discussed above, a body in motion can produce background noise that obscures at least some of the data indicating contraction of a target muscle. Analysis can compensate for the presence of background noise by using additional myography sensors in order to make a background noise model which is used to remove at least some of the actual background noise. Analysis can also compensate for the presence of background noise by one or a combination of removing a background noise model from frequency data (such as by using at least one myography sensor located away from the target myography sensor) and gathering biomechanical motion data (such as by using at least one motion capture device) in order to determine which muscles are likely to be under contraction and to target and better identify signals of interest during myography, which can be muscle vibrations signals, muscle electrical signals, or muscle acoustic signals.

The biomechanics of body movement can be used to infer what muscles are likely to be activated and/or determine how the muscle is working if it is active. For example, it is likely, but not guaranteed, that a person's biceps muscle is activated when a straight arm bends so that the hand curls toward the shoulder. This and other biomechanical information (e.g., information regarding movement of a foot, leg, back, abdomen, neck, etc.) can be used to infer what muscle is likely to be activated among multiple muscles that are being monitored. By knowing what muscle is likely to be activated, the analysis can be target toward data obtained from the myography sensor at or near the muscle that is deemed likely to be activated. Targeting the analysis allows the system to try harder to detect a potential signal useful for myography.

Detecting biomechanics of a body movement can also allow determination of whether a muscle is potentially in concentric contraction (muscle is active and pulling shorter), isometric contraction (muscle is active but remaining the same length), or eccentric contraction (muscle is active but is being pulled longer). For example, the type of contraction that a biceps muscle is undergoing can be determined by detecting whether a person's hand is moving towards or away from her shoulder. Concentric contraction of the biceps muscle is likely to be occurring when the hand is moving towards the shoulder. Eccentric contraction of the biceps muscle is likely to be occurring when the hand is moving away from the shoulder. Muscles can have different vibrational signatures which are specific to the type of contraction. Thus, with knowledge of the type of contraction that is likely to be occurring, the data from the appropriate sensor can be analyzed by the system to look for the specific signature of the appropriate type of contraction.

For example, the signal for concentric muscle contraction may be dominated by high energy frequencies (referred to herein as “dominant frequencies”) which are not dominant in signals for isometric and/or eccentric contraction. The signature for concentric muscle contraction would include the combination dominant frequencies unique to concentric muscle contraction. The signature of isometric muscle contraction would include a different combination of dominant frequencies unique to isometric muscle contraction. The signature of eccentric muscle contraction would include yet another combination of dominant frequencies unique to eccentric muscle contraction. The various signatures may have some dominant frequencies in common. These signatures (e.g., unique combinations of dominant frequencies) could be known in advance of obtaining frequency data from myography sensors. If concentric contraction is determined to be likely to be occurring, a processor will narrow the analysis to look at frequencies at the dominant frequencies for concentric contraction in order to confirm that the likely muscle is actually undergoing contraction and that the contraction is actually concentric. Narrowing the analysis can be similarly performed for isometric contraction and eccentric contraction.

Detecting biomechanics of a body movement can be performed in various ways. For example, a system can perform real-time motion capture using motion capture sensors which may include without limitation, video cameras, a depth camera, multiple body-worn sensors, or any combination thereof. Skeletal movement is modeled or obtained by the system. The skeletal model includes relationships between body parts: the foot is a child of the shin, the shin is a child of the upper leg, the upper leg is a child of the hip, and so on. The skeletal model further includes known muscle functions. Input from one or more motion capture sensors is combined with the skeletal model to produce a model of biomechanical movement which identifies the muscles that are most likely to be driving the body motion. For example, quadriceps muscles are known to be used to straighten a leg. When a motion capture sensor detects that a person's left leg is moving from bent to straight, the left quadriceps muscle is identified as a muscle that is likely to be activated.

The muscle that is likely to be activated is referred to herein as the “likely muscle” for brevity.

Myography may involve a “full-body” system having many sensors being placed all over the body on many muscles. A full body system can take a huge amount of computing resources. Input from motion capture sensors can identify likely muscles and muscles which are not likely to be activated. Identification of likely muscles can be used to optimize myography by directing potentially scarce computing resources toward evaluation of data from only the sensors resting on or near likely muscles. Also, the likelihood of muscle activation of a particular muscle (e.g., as a primary, secondary, or tertiary driver of the motion) may be used to weight an algorithm that detects and/or analyzes muscle activity.

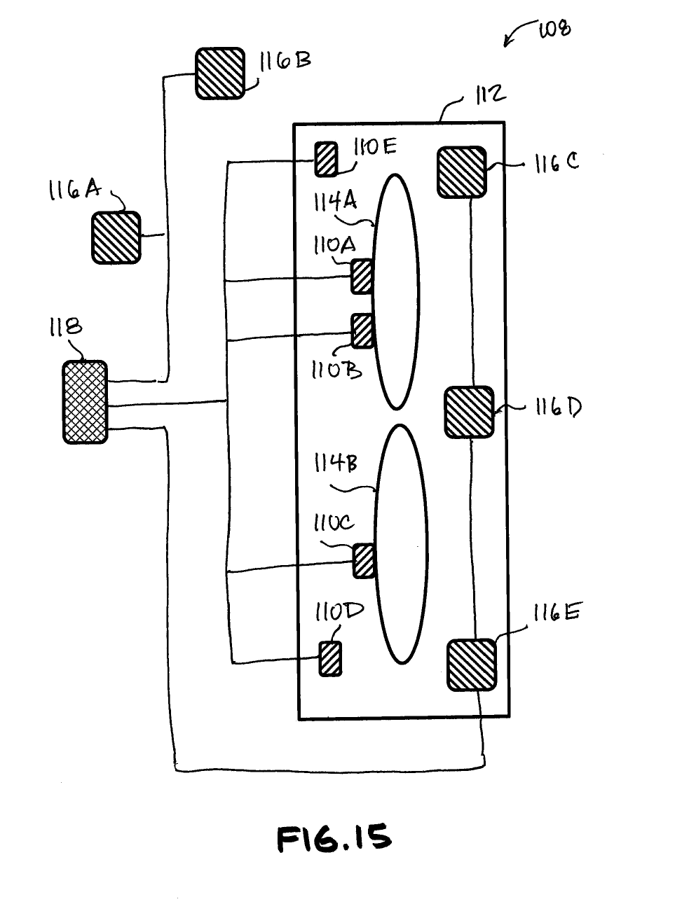

FIGS. 14 and 15 illustrate an exemplary method and system for targeting analysis of muscle activity from biomechanical motion data. As shown in FIG. 15, system 108 includes myography sensors 110A-110E attached to body 112. Myography sensors 110A-110C are placed on target muscles 114A and 114B. System 108 includes motion capture sensors 116A-116D configured to detect biomechanical motion. Motion capture sensors 116A and 116B are optical imaging devices, such as video cameras located at a distance away from body 112. Motion capture sensors 116C and 116D are on-body sensors (i.e., sensors attached to body 112), which can be similar to or the same as inertial measurement units 10 in FIGS. 2 and 3. Motion capture sensors 116C and 116D on body 112 may also be configured to function as myography sensors to detect signals indicative of muscle activation. Any one or a combination of motion capture sensors 116A-116D can be used by processor 118 to detect biomechanical motion. Processor 118 is communicatively coupled to myography sensors 110A-110E and motion capture sensors 116A-116D.

The process steps of FIG. 14 can be performed by processor 118. Processor 118 can be configured like processor 67 in FIG. 7. Processor 118 can include one or more semiconductor chips. Processor 118 can be part of an electronic computer or group of computers capable of executing, in accordance with a computer program stored on a non-transitory computer readable medium, any one or a combination of the process steps of FIG. 14. The non-transitory computer readable medium may comprise instructions for performing any one or a combination of the process steps of FIG. 14.

In block 120 of FIG. 14, acceleration data of vibrations are collected by processor 118 from myography sensors 110A-110E and optionally from any motion capture sensor 116C and 116E which may be configured to detect vibrations indicative of muscle contraction. The acceleration data is collected for periods of time to acquire sufficient data that will allow frequency analysis to be performed. The period of time could, for example, be half second with samples taken every few milliseconds, or less than one second with samples taken every few milliseconds. Other time periods and sampling rates may be used.

In block 122, a Fast Fourier Transform (FFT) is performed by processor 118 on the acceleration data obtained from block 120 to produce frequency data representing what frequencies of vibration were observed and the amount of energy at each frequency.

In block 124, background energy is optionally removed by processor 118 from the frequency data obtained from block 122. If background energy is to be removed from the frequency data, the process steps of blocks 84, 86, and 88 in FIG. 12 can be performed by processor 118.

In block 126, biomechanical motion is captured by any one or more of motion capture sensors 116A-116E while the acceleration data is collected in block 120.

In block 128, motion capture data from block 126 is used by processor 118 to create a biomechanical model of movement of body 112. The biomechanical model identifies the body joints (e.g., a knee, hip, and ankle joints) associated with target muscles 114 (e.g., hamstring and calf muscles in a leg) and identifies the direction (e.g., straightening versus bending) that the body joints are presently moving.

In block 130, the present direction of movement associated with each target muscle 114 is identified by processor 118 based on the biomechanical model created by processor 118 in block 128.

In blocks 132 and 134, for each target muscle 114, processor 118 uses the biomechanical model and information on which muscles 114 are likely to be activated to determine whether the muscle is lengthening (which is associated with eccentric muscle contraction), shortening (which is associated with concentric muscle contraction), or staying at the same length (which is associated with isometric muscle contraction).

According to block 132, if target muscle 114 is shortening, the method proceeds to block 136. According to block 134, if target muscle 114 is staying at the same length, the method proceeds to block 138. If target muscle 114 is not shortening and not staying at the same length, then target muscle 114 is lengthening and the method proceeds to block 140.

In block 136, acceleration (vibrational) data is analyzed in the frequency domain by processor 118 by looking for the vibrational signature expected for concentric muscle contraction. For example, if target muscle 114A is determined from the biomechanical model to be lengthening, processor 118 analyzes frequency data from myography sensors 10A and 10B by looking for possible concentric muscle contraction.

In block 138, acceleration (vibrational) data is analyzed in the frequency domain by processor 118 by looking for the vibrational signature expected for isometric muscle contraction. For example, if target muscle 114A is determined from the biomechanical model to be staying at the same length, processor 118 analyzes frequency data from myography sensors 10A and 10B by looking for possible isometric muscle contraction.

In block 140, acceleration (vibrational) data is analyzed in the frequency domain by processor 118 by looking for the vibrational signature expected for eccentric muscle contraction. For example, if target muscle 114B is determined from the biomechanical model to be lengthening, processor 118 analyzes frequency data from myography sensor 10C by looking for possible eccentric contraction.

In the examples of FIGS. 12-15, the myography sensors were configured to detect vibrations for the purpose of performing mechanomyography. Those sensors can be replaced with electromyography sensors configured to detect electrical signals from muscles for the purpose of performing electromyography or replaced with acoustic myography sensors configured to detect acoustic signals for the purpose of performing acoustic myography.

The steps of FIGS. 12 and 14 may be repeated as desired. Conditions external and internal to the moving body may change with each repetition so that the model of background noise may differ from the previous model, the analysis (for concentric, isometric, or eccentric contraction) performed for a muscle may differ from the analysis performed previously on that muscle, and/or the analysis may be performed on a muscle that was not previously analyzed. Thus, it will be appreciated that the methods and systems for myography described herein are capable of adapting to changing conditions presented by a moving body. By correlating observed likely muscle activity and observed myography readings, a system can learn the frequency signatures of different muscles.

As indicated above, a computer readable medium may comprise any one or a combination of instructions for removing background energy, for inferring what muscles are likely to be activated, and for determining whether a muscle is under concentric, isometric, or eccentric contraction. Suitable examples of such a computer readable medium include without limitation optical storage devices (e.g., a CD-ROM), magnetic storage devices (e.g., hard disk drive), and flash memory devices (e.g., memory cards). Other forms of computer readable media known in the art may be used.

It will be appreciated from the descriptions herein that the myography methods and systems of the present invention can be used on a body in motion by compensating for background noise in various ways. As used herein, the phrase “body in motion” refers to motion that includes both motion of the target muscle(s) which produce(s) signals of interest in myography and motion of other parts of the body which produce signals not of interest in myography. For example, the myography methods and systems can be used on a body in which the target muscle is in the left arm while the right arm is moving and while the person remains at a stationary location on the ground. As a further example, the myography methods and systems can be used on a body in which the target muscle is in the left arm while the person is walking or running from one location to another. Motion at parts of the body other than the target muscle produces at least some of the background noise. The background noise can include noise produced by the environment surrounding the body and by other sources.

Compensating for background noise can include, without limitation, one or a combination of steps described above for making a model of the background noise, for removing at least some of the actual background noise using a model of the background noise, for identifying muscles likely to be under contraction from among a plurality of muscles that are being monitored for possible contraction, and for narrowing the analysis to searching for the type of muscular contraction (e.g., concentric, isometric, or eccentric) that is likely to be occurring.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. A myography method comprising:
obtaining frequency data from each of a plurality of myography sensors on a body in motion, wherein the myography sensors include a target myography sensor at a target muscle on the body and one or more myography sensors located at other parts of the body, the motion of the body produces background noise, and the frequency data from the target myography sensor includes the background noise and data on muscle contraction of the target muscle; and
analyzing the data on muscle contraction of the target muscle, including compensating for the background noise.

2. The method of claim 1, wherein the compensating for background noise includes removing a model of the background noise from the frequency data of the target myography sensor.

3. The method of claim 2, wherein the compensating for background noise includes making the model of the background noise from the frequency data of the one or more myography sensors located at other parts of the body.

4. The method of claim 3, wherein the making of the model of the background noise includes comparing the frequency data from the plurality of myography sensors for commonality.

5. The method of claim 4, wherein the comparing includes searching for high energy frequencies occurring in common in the frequency data of at least some of the plurality of myography sensors.

6. The method of claim 1, wherein the compensating for the background noise includes identifying which of the plurality of myography sensors are at a muscle likely to be undergoing contraction.

7. The method of claim 6, wherein the identifying includes making a model of biomechanical movement of the body and using the model of biomechanical movement to identify which of the plurality of myography sensors are at a muscle undergoing contraction.

8. The method of claim 7, wherein the making of the model of biomechanical movement includes obtaining motion capture data from one or more motion capture devices configured to detect motion of the body.

9. The method of claim 6, wherein the compensating for the background noise includes determining a type of muscular contraction for each myography sensor identified as likely to be undergoing contraction.

10. The method of claim 9, wherein the compensating for the background noise includes looking, in frequency data of each myography sensor identified as likely to be undergoing contraction, for a signature of the determined type of muscular contraction.

11. A myography method comprising:
obtaining frequency data from one more myography sensors on a body in motion, the frequency data including myography data on muscle contraction of a target muscle;
making a model of biomechanical movement while obtaining the frequency data; and
analyzing the myography data with a heuristic for the analysis being different as a function of what the model of biomechanical movement indicates the target muscle is likely doing.

12. The method of claim 11, wherein the heuristic for analysis of the myography data depends on any one or a combination of (a) whether the model of biomechanical movement indicates the target muscle is contracting and (b) whether the model of biomechanical movement indicates the target muscle is not contracting.

13. The method of claim 11, further comprising changing the heuristic for analysis of the myography data according to any one or a combination of (a) whether the model of biomechanical movement indicates the target muscle is contracting and (b) whether the model of biomechanical movement indicates the target muscle is not contracting.

14. The method of claim 11, wherein the heuristic for analysis of the myography data depends on any one or a combination of (a) whether the model of biomechanical movement indicates the target muscle is working by concentric contraction, (b) whether the model of biomechanical movement indicates the target muscle is working by isometric contraction, and (c) whether the model of biomechanical movement indicates the target muscle is working by eccentric contraction.

15. The method of claim 11, further comprising changing the heuristic for analysis of the myography data according to on any one or a combination of (a) whether the model of biomechanical movement indicates the target muscle is working by concentric contraction, (b) whether the model of biomechanical movement indicates the target muscle is working by isometric contraction, and (c) whether the model of biomechanical movement indicates the target muscle is working by eccentric contraction.

16. The method of claim 11, wherein the heuristic is different, as a function of what the model of biomechanical movement indicates the target muscle is likely doing, in terms of any one or a combination of (a) frequencies of muscle vibrations to indicate a likely muscle contraction and (b) thresholds for amounts of energy present at frequencies of muscle vibrations to indicate a likely muscle contraction.

17. The method of claim 11, further comprising changing the heuristic for analysis of the myography data in terms of any one or a combination of (a) frequencies of muscle vibrations to indicate a likely muscle contraction and (b) thresholds for amounts of energy present at frequencies of muscle vibrations to indicate a likely muscle contraction.

18. The method of claim 11, wherein the making of the model of biomechanical movement includes obtaining motion capture data from one or more motion capture devices configured to detect motion of the body.

19. The method of claim 11, further comprising making a model of background noise and removing the model of background noise from the frequency data.

20. A myography system comprising:
a plurality of myography sensors; and
a processor configured to obtain frequency data from each of the plurality of myography sensors while the myography sensors are on a body in motion, the processor further configured to compensate for background noise produced by body motion to analyze data on muscle contraction from a target muscle.

21-29. (canceled)

30. A non-transitory computer readable medium having a stored computer program embodying instructions, which when executed by a computer, causes the computer to perform myography, the computer readable medium comprising:
instructions for obtaining frequency data from each of a plurality of myography sensors on a body in motion, wherein the myography sensors include a target myography sensor at a target muscle on the body and one or more myography sensors located at other parts of the body; and
analyzing data on muscle contraction of the target muscle, including compensating for background noise which is produced by the motion of the body and is present in the frequency data together with the data on muscle contraction.

31-39. (canceled)
